(19) United States
(12) Patent Application Publication
(10) Pub. No.: US 2022/0110778 A1
(43) Pub. Date: Apr. 14, 2022

(54) OSTOMY BAG EVACUATION CONTAINMENT MECHANISM AND METHOD OF USING

(71) Applicant: Mikhail N. Levin, Troy, NY (US)

(72) Inventor: Mikhail N. Levin, Troy, NY (US)

(21) Appl. No.: 17/068,743

(22) Filed: Oct. 12, 2020

(51) Int. Cl. A61F 5/44; A61F 5/449

(52) U.S. Cl. CPC A61F 5/4408; A61F 5/449

(57) ABSTRACT

An ostomy bag emptying device comprising an ostomy bag with a sealed top end and a bottom end, said ostomy bag being attached by a commercially available means to a patient at a stoma site, an optional belt holding said ostomy bag in place, an external receptacle with a top, a bottom, a body side and an outer side; and a suspension mechanism of fixed or adjustable length by which said external receptacle is suspended at a stoma site from said belt is taught, together with a method of using said ostomy bag emptying device.

### BACKGROUND OF THE INVENTION

### 1. Field of the Invention

This invention lies in the field of ostomy equipment and its use, specifically the containment and disposal of effluvium evacuated from an ostomy bag.

### 2. Description of Related Art

A patient may need an ostomy, or the surgical introduction of a hole in the body wall, for a variety of reasons. The patient may have colon cancer, celiac disease, or a variety of other diseases or disorders wherefor the removal of part of the intestine is the recommended procedure. Removal of part of the intestine renders the rectum inoperative, requiring another path for fecal material and gas to exit the body. In a urostomy patient, the urinary bladder is damaged or defective in some way, requiring another path for urine to exit the body. The exit site in either case is known as a stoma.

Management of the effluvium of fecal and gas or urine from the body is accomplished with an ostomy bag. Ostomy bags are well known in the art. An ostomy bag may be required with any stoma, but is most frequently used with urostomy, ileostomy, and colostomy stomas to contain bodily waste until the patient can conveniently evacuate and clean the ostomy bag.

Ostomy bags may be drainable or not drainable. Ostomy bags, specifically drainable ostomy bags, come in many varieties; some have loops for attachment to a belt or waistband while others do not.

Ostomy flanges that attach to the body and hold the ostomy bag securely in place at the stoma site may be integral with the ostomy bag or separate. Ostomy bags may have belt loops that hold an ostomy bag belt or no loops, in which case no ostomy bag belt is used.

Ostomy patients face the problem of comfortably, cleanly, safely and completely evacuating the ostomy bag when the bag fills with effluvium. This issue is fairly easily dealt with at home through the use of home-based equipment that is well-known in the art, but the problem becomes acute when the patient is away from home and away from their home-based equipment. At such times, the comfortable, clean, safe evacuation and disposal of ostomy bag contents can be very difficult, especially if the ostomy patient has any physical disability such as arthritis, obesity, old age, disease, or other physical limitation. The ostomy patient must assume uncomfortable postures to empty the ostomy bag directly into a toilet, and may splash the contents of the ostomy bag in a public restroom, where such splashing may endanger the health of the patient and require cleaning of the toilet and stall before the toilet and stall can be safely used by others.

Emptying a drainable ostomy bag involves releasing a valve or pinched fold at the base of the ostomy bag and allowing the effluvium contained therein to drain out of the ostomy bag, usually into a rigid container such as a bucket or toilet. However, the process of draining the effluvium into a toilet or bucket can result in the patient needing to assume uncomfortable or even unstable positions, and in splashing the effluvium back out of the toilet or bucket and onto the patient. This is unpleasant and unsanitary for the patient and for the public facility.

Roche, in U.S. Pat. No. 7,993,313, teaches “An apparatus to receive feces from an ostomy pouch or urine from a person for disposal which comprises an adjustable strap member worn about a neck of the person to hang down therefrom. A waste receptacle is provided. A mechanism is for detachably mounting the waste receptacle to distal ends of the adjustable strap member at a groin area of the person. The person can discharge the feces from an open end of the ostomy pouch and also urinate into the waste receptacle.” Roche's invention, however, is inconvenient for the patient in that the waste receptacle hangs from the patient's neck. This is unwieldy to carry and the patient cannot bend or twist without affecting the position of the waste receptacle relative to the stoma site.

### SUMMARY OF THE INVENTION

The invention comprises an effluvium collection system that is worn directly at the ostomy site and comfortably, conveniently, cleanly discretely, and completely allows for evacuation of the ostomy bag when the patient is not at home.

The invention comprises an ostomy bag with a sealed top end and a bottom end with a stopper, said ostomy bag being attached by a commercially available means to a patient directly at a stoma site, a leakproof external receptacle with a top, a bottom, a body side, an outer side and a lumen; and a suspension mechanism by which said leakproof external receptacle is removably affixed near said patient's stoma site.

The leakproof external receptacle may be a clean flushable ostomy bag liner, a thin-membrane plastic bag such as is commonly used to contain groceries, a bag commonly found on airplanes used for containing vomitus, a plastic bag commonly used for food storage that is closed with a tying mechanism, and a plastic bag commonly used for food storage that is closed with a zipper mechanism.

The suspension mechanism may consist of garters, grommets, string, yarn, thread, pins, adhesive, loops, loops integral to said leakproof external receptacle, hooks, hook-and-loop systems such as VELCRO®, or snaps. It may be of fixed length or adjustable length.

The bottom end of the ostomy bag is removably placed within the lumen of a leakproof external receptacle prior to emptying the ostomy bag, such that the contents of the ostomy bag are deposited directly into the leakproof external receptacle.

The method of using the ostomy bag emptying device requires the patient to place the suspension mechanism on a belt on either side of an ostomy bag. Alternatively, the patient may place the suspension mechanism directly on the belt holders on the ostomy bag.

The patient then attaches a leakproof external receptacle to the suspension mechanism, placing the bottom end of the ostomy bag within the lumen of the leakproof external receptacle.

When the ostomy bag is ready to empty, the patient releases a stopper mechanism on the bottom end of the ostomy bag to allow the flow of effluvium from the ostomy bag into the leakproof external receptacle, thus filling said leakproof external receptacle with effluvium from the ostomy bag. When the leakproof external receptacle is filled, the patient closes the stopper and removes the bottom end of the ostomy bag from the leakproof external receptacle, then removes the filled leakproof external receptacle from the suspension mechanism. The patient can then dispose of the filled leakproof external receptacle and its contents in a safe, comfortable, and sanitary way.

The patient can then replace said leakproof external receptacle with a clean leakproof external receptacle.

One advantage of the present invention over other inventions in the art is the patient can move in any manner (bending, twisting) without altering the position of said leakproof external receptacle relative to said ostomy bag.

### BRIEF DESCRIPTION OF THE DRAWINGS

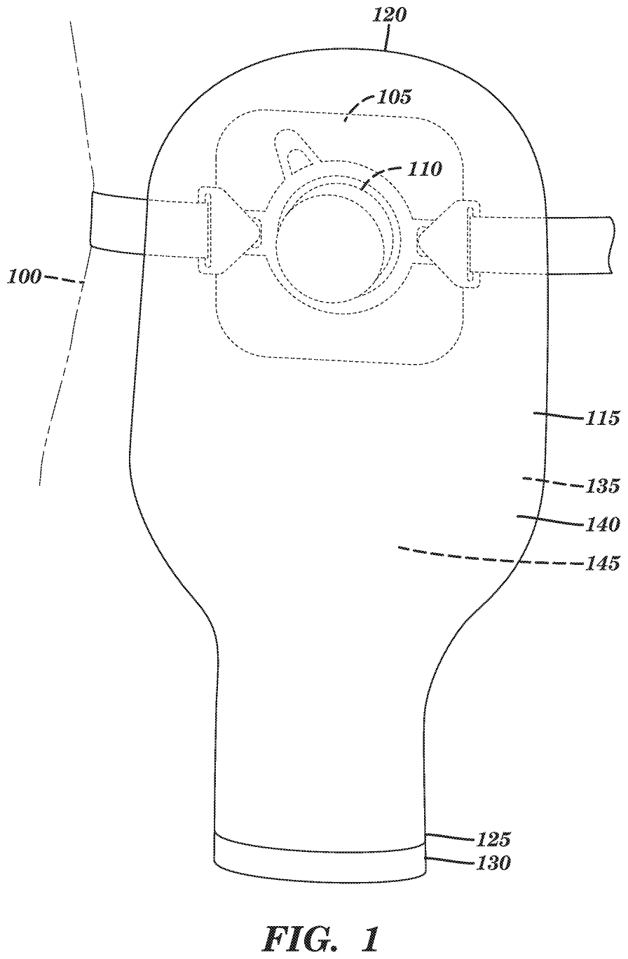

Referring now to FIG. 1, a flange that attaches to the patient's body for holding a ostomy bag in place at a stoma site is shown.

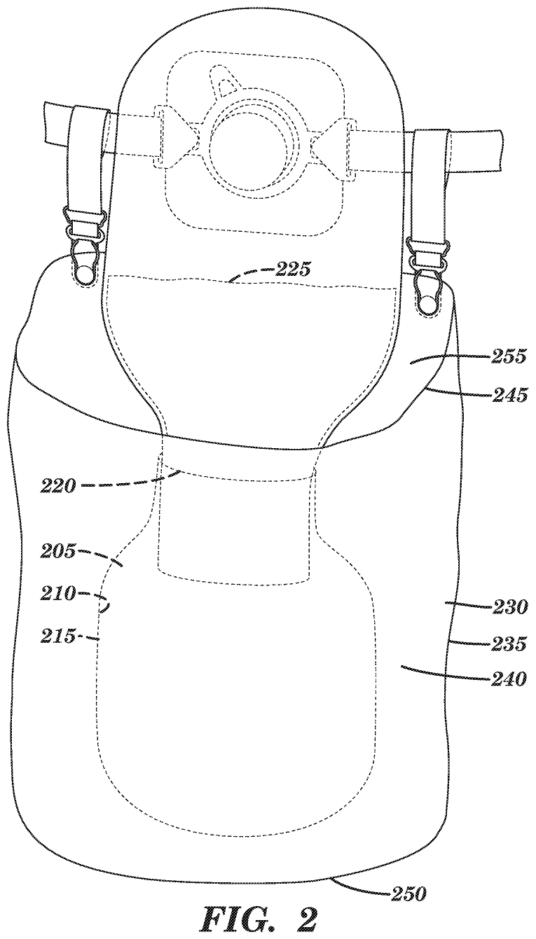

Referring now to FIG. 2, a ostomy bag portable emptying apparatus is shown.

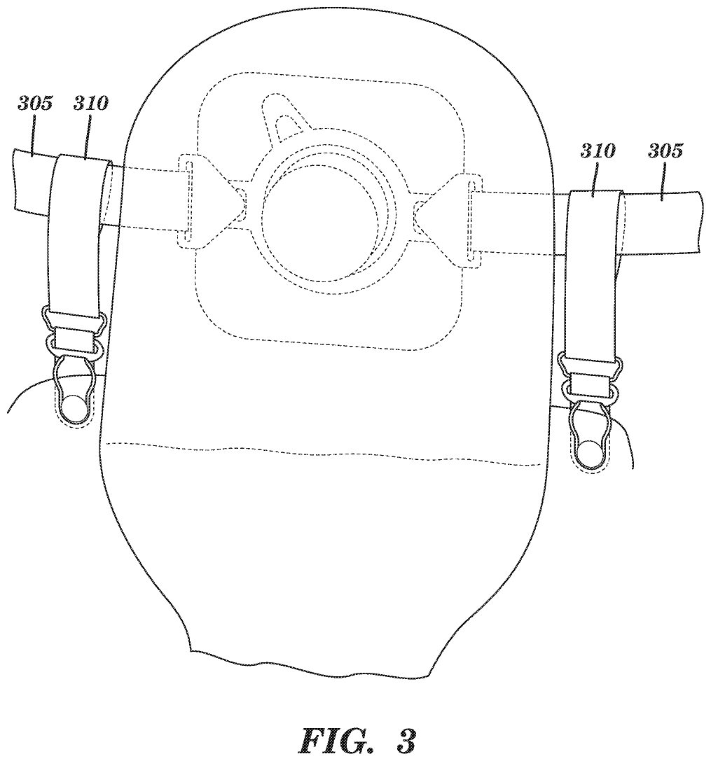

Referring now to FIG. 3, an apparatus for holding a ostomy bag portable emptying apparatus in place is shown.

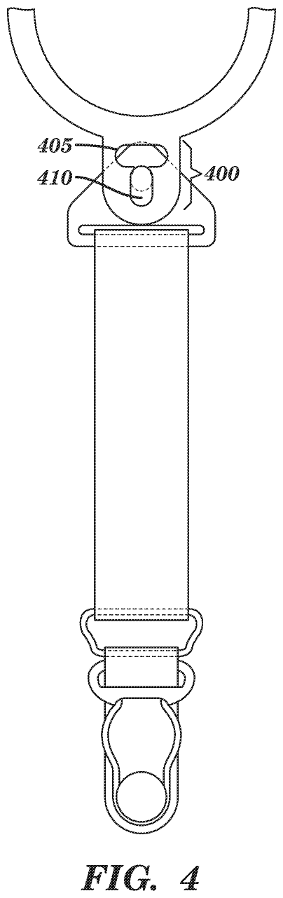

Referring now to FIG. 4, an alternate means of attaching the ostomy bag portable emptying apparatus to the patient's stoma site is shown.

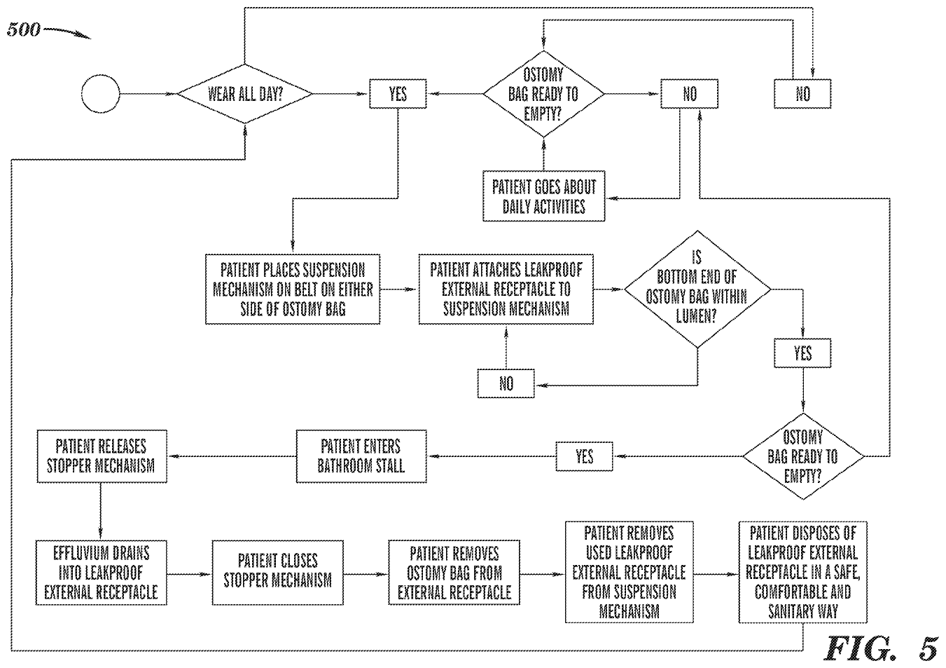

Referring now to FIG. 5, a method of using said apparatus for holding a ostomy bag portable emptying apparatus is shown.

### DETAILED DESCRIPTION OF THE DRAWINGS

Referring now to FIG. 1, a patient 100 must attach a flange 105 to the stoma site 110 to anchor a commercially available ostomy bag 115 in place. The ostomy bag may be a drainable bag or a non-drainable bag. Said ostomy bag 115 has a sealed top end 120 and a bottom end 125, said bottom end 125 comprising a stopper mechanism 130 which may be a commercially available stopper mechanism including valves, clip, hook-and-loop connectors such as VELCRO®, or simply folding or pinching of said ostomy bag 115, Said ostomy bag 115 has an inside 135 and an outside 140, surrounding an ostomy bag lumen 145. Such ostomy bag 115, flange 105, and stopper mechanism 130 are well known in the art, are commercially available, and are not contemplated as a part of this invention, although they are necessary for the invention to be effective.

Referring now to FIG. 2, said ostomy bag lumen 145 contains effluvium 225 that is discharged from said stoma site 110. When said ostomy bag lumen 145 is filled, said patient 100 must empty said ostomy bag 115. When at home or in another environment in which supplies and equipment can be stored, said patient 100 can easily empty said effluvium 225 into a rigid container such as a toilet or bucket. However, when said patient is in a public restroom away from stored supplies, the ability to use said rigid container is generally unavailable; said use even at home may actually be unsafe for a patient 100 with physical limitations such as arthritis, obesity, age, or disease. At such times, use of a portable leakproof receptacle 230 is by far the better solution. Said leakproof receptacle comprises at least two sides 235, an open top 245, a closed bottom 250, and a lumen 255, said lumen being the space found between said sides 235.

Said patient 100 inserts said ostomy bag bottom end 125 through said portable leakproof receptacle open top 245 into said portable leakproof receptacle lumen 255 and releases said stopper mechanism 130, at which time said effluvium 225 passes out of said ostomy bag 115 through said bottom end 125 into said portable leakproof external receptacle lumen 255.

After said ostomy bag 115 is emptied, said patient 100 reseals said bottom end 125.

Said portable leakproof external receptacle 230 allows said patient 100 to empty said effluvium in a comfortable, safe and sanitary manner, without danger of splashing and without the need to position said patient's body in an uncomfortable or unsafe position.

Alternatively, said ostomy bag 115 in a commercially available configuration may contain an optional ostomy bag liner 205 which has at least two ostomy bag liner inner sides 210, at least two ostomy bag liner outer sides 215, and an optional ostomy bag liner lumen 220, said optional ostomy bag liner lumen 220 being located between said optional ostomy bag liner inner sides 210. While the use of said optional ostomy bag liner 205 in an ostomy bag 115 as known in such a commercially available configuration is not a recommended configuration of the present invention, the patient 100 may benefit by using said optional ostomy bag liner 205 as a liner for said portable leakproof external receptacle 230 very effectively.

Said patient 100 may also use said optional ostomy bag liner 205 within said ostomy bag 115 and remain within the scope of the present invention, but such a configuration is not preferred and is not pictured here.

Referring now to FIG. 3, said ostomy bag 115 is attached to said patient 100 using said flange 105, which attaches to said patient 100 using a medical-grade adhesive. In the embodiment shown, a belt 305 encircles said patient's 100 waist. Said belt 305, in some embodiments, may be used to support said ostomy bag 115; in other embodiments, said belt 305 functions simply to hold clothing in place. Said suspension mechanism is attached at the same point on the body as the bag and is therefore unaffected by bodily movement or by the patient's 100 body shape.

In all embodiments, said leakproof external receptacle 230 for effluvium 225 is suspended from said belt 305 using a suspension mechanism 310. The present embodiment contemplates garters being used as said suspension mechanism 310, but other suspension mechanisms 310, including grommets, string, yarn, thread, pins, adhesive, loops, loops integral to said leakproof external receptacle 230, hooks and eyes, hooks alone, and snaps are also possible and are considered to be part of the present invention.

Said suspension mechanism 310 straddles said ostomy bag 115, such that said bottom end 125 of said ostomy bag 115 is inserted into said leakproof bag lumen 255. When said bottom end 125 is so inserted into said leakproof bag lumen 255, said patient 100 can easily release said stopper mechanism 130 to allow said effluvium 225 to drain into said leakproof bag lumen 255. When draining is complete, said patient 100 disposes of said leakproof external receptacle 230 in a comfortable, easy and sanitary manner. Said patient 100 then re-engages said stopper mechanism 130 to allow said ostomy bag lumen 145 to collect effluvium 225.

In the present embodiment, said leakproof external receptacle 230 comprises a flushable ostomy bag liner 205 held in place by said suspension mechanism 310. In another embodiment, said leakproof external receptacle 230 comprises a member of the set consisting of a flushable ostomy bag liner 205 thin-membrane plastic bag such as is commonly used to contain groceries, a bag commonly found on airplanes for containing vomitus commonly used for airsickness, a plastic bag commonly used for food storage that is closed with a tying mechanism, and a plastic bag commonly used for food storage that is closed with a zipper mechanism.

Referring now to FIG. 4, an alternate suspension mechanism 400 is shown wherein said alternate suspension mechanism 400 is suspended directly from a belt attachment loop 405 on said ostomy bag 115. Said alternate suspension mechanism 400 is fitted with a hook 410 that attaches to said belt attachment loop 405 to suspend said alternate suspension mechanism 400 directly from said ostomy bag 115. Note that FIG. 4 represents only one of many commercially available embodiments, the use of each of which is contemplated as part of the present invention.

Referring now to FIG. 5, a process 500 whereby said patient 100 empties said ostomy bag 115 is shown. Said patient 100 starts the process by deciding whether to wear said suspension mechanism 310 throughout daily activities. If said patient 100 decides to wear said suspension mechanism 310 throughout daily activities, said patient 100 must then decide whether to wear said external leakproof external receptacle 230 throughout daily activities,

Should said patient 100 decide not to wear said suspension mechanism 310 throughout daily activities, said patient 100 must then attach said suspension mechanism 310, together with said leakproof external receptacle 230, to said belt 305 or said hook 410 immediately prior to emptying said ostomy bag 115.

Should said patient decide to wear said suspension mechanism 310 but not said external leakproof external receptacle 230 throughout daily activities, said patient 100 must then affix said leakproof external receptacle 230 to said suspension mechanism 310 immediately prior to emptying said ostomy bag 115. Said patient 100 attaches said leakproof external receptacle 230 to said suspension mechanism 310 when attaching said ostomy belt 305 to said belt attachment loop 405. Said patient 100 places said suspension mechanism 310 on said belt 305 in such a way that said suspension mechanism 310 is located on either side of said ostomy bag 115.

Alternatively, said patient 100 may hang said hook 410 of said suspension mechanism 310 from said belt attachment loop 405.

Said patient 100 then attaches said leakproof external receptacle 230 to said suspension mechanism 310 such that said bottom end 125 is within said leakproof bag lumen 255.

Said patient 100 can go about his or her daily activities while wearing said leakproof external receptacle 230, waiting for said ostomy bag 115 to require emptying. In another embodiment, said patient 100 need not wear said leakproof external receptacle 230 while going about his or her daily activities, but must attach said leakproof external receptacle 230 to said suspension mechanism 310 at the time said patient 100 empties said ostomy bag 115. When said leakproof external receptacle 230 is ready to empty, said patient 100 enters a bathroom stall and releases said stopper mechanism 130. Said effluvium 225 drains from said ostomy bag liner 205 into said leakproof external receptacle 230. Said patient 100, when said effluvium 225 is entirely drained, can then close said stopper mechanism 130, remove said leakproof external receptacle 230 from said suspension mechanism 310, dispose of said used leakproof external receptacle 230 in a sanitary way, replace said used leakproof external receptacle 230 with an empty leakproof external receptacle 230, and proceed with daily activities.

In another embodiment, said patient 100 replaces said leakproof external receptacle 230 at the next time said patient 100 needs to empty said ostomy bag 115 rather than wearing said leakproof external receptacle 230 until it again becomes necessary to empty said ostomy bag 115.

In another embodiment, said optional flushable liner 205, which is easily torn, provides a lining to a sturdier leakproof external receptacle 230 (making cleaning said leakproof external receptacle 230 significantly easier and faster for said patient 100) wherein said leakproof external receptacle 230 is affixed to said suspension mechanism 310. Said effluvium 225 collects in said optional flushable liner 205, and said patient 100 can flush the optional flushable liner 205. However, when flushable liner 205 is not used, said patient 100 must also dispose of said external receptacle 230 using a conventional trash container, which is less discrete than is the use of said optional flushable liner 205.

The embodiments provided here are examples only and are not intended to be a complete listing of possible embodiments, nor should they be construed as an exclusive listing of embodiments. Variations in the described invention and its uses are possible within the scope of this disclosure without departing from the subject matter coming within the scope of claims to be examined, and a reasonable equivalency thereof, which I regard as my invention.

I claim:

1. An ostomy bag emptying device comprising:
a commercially available ostomy bag with a sealed top end and a bottom end with a stopper, said ostomy bag being attached by a commercially available means to a patient directly at a stoma site;
a leakproof external receptacle with a top, a bottom, a body side, an outer side and a lumen; and
a suspension mechanism by which said leakproof external receptacle is removably affixed near said patient's stoma site.

2. The leakproof external receptacle of claim 1 wherein said leakproof external receptacle is chosen from the group consisting of a clean flushable ostomy bag liner, a thin-membrane plastic bag such as is commonly used to contain groceries, a bag commonly found on airplanes used for containing vomitus, a plastic bag commonly used for food storage that is closed with a tying mechanism, and a plastic bag commonly used for food storage that is closed with a zipper mechanism.

3. The suspension mechanism of claim 1 wherein said suspension mechanism is selected from the group consisting of garters, grommets, string, yarn, thread, pins, adhesive, loops, loops integral to said leakproof external receptacle, hooks, hook-and-loop systems and snaps.

4. The suspension mechanism of claim 1 wherein the length of said suspension mechanism is selected from the group consisting of fixed and adjustable.

5. The ostomy bag emptying device of claim 1 wherein a bottom end of an ostomy bag is removably placed within a leakproof external receptacle lumen.

6. A method of using an ostomy bag emptying device comprising:
A patient placing a suspension mechanism on a belt on either side of an ostomy bag;
Said patient attaching a leakproof external receptacle to said suspension mechanism such that a bottom end of said ostomy bag is within a lumen of said leakproof external receptacle;
When said ostomy bag is ready to empty, said patient releasing a stopper mechanism to allow the flow of effluvium from said ostomy bag into said leakproof external receptacle, thus filling said leakproof external receptacle with effluvium from said ostomy bag;
When said leakproof external receptacle is filled, said patient closing said stopper and removing said bottom end of said ostomy bag from said leakproof external receptacle;
Said patient removing said filled leakproof external receptacle from said suspension mechanism;
Said patient disposing of said filled leakproof external receptacle and its contents in a safe, comfortable and sanitary way;
Said patient replacing said leakproof external receptacle with a clean leakproof external receptacle.
Said patient moving in any manner does not alter the position of said leakproof external receptacle in relation to said ostomy bag.

7. The method of claim 5 further comprising said leakproof external receptacle being chosen from the group consisting of a clean flushable ostomy bag liner, a thin-membrane plastic bag such as is commonly used to contain groceries, a bag commonly found on airplanes used for containing vomitus, a plastic bag commonly used for food storage that is closed with a tying mechanism, and a plastic bag commonly used for food storage that is closed with a zipper mechanism.

8. The method of claim 5 further comprising said suspension mechanism being chosen from the group consisting of garters, grommets, string, yarn, thread, pins, adhesive, loops, loops integral to said leakproof external receptacle, and snaps.
